SEQUENCE LISTING (7 sequences)

SEQ ID NO: 1 - protein, 631 aa, Hepatitis C virus
  MISC_FEATURE (1)..(631): Non-structural NS3 protein.
APITAYAQQTRGLLGCIITSLTGRDKNQVEGEVQIVSTAAQTFLATCINGVCWTVYHGAGTKTIASSKGPVIQMYTNVDQDLVGWPAPQGARSLTPCTCGSSDLYLVTRHADVIPVRRRGDSRGSLLSPRPISYLKGSSGGPLLCPAVHAVGIFRAAVCTRGVAKAVDFIPVEGLETTMRSPVFSDNSSPPAVPQSYQVAHLHAPTGSGKSTKVPAAYAAQGYKVLVLNPSVAATLGFGAYMSKAHGIDPIIRTGVRTITTGSPITYSTYGKFLADGGCSGGAYDIIICDECHSTDATSILGIDTVLDQAETAGARLTVLATATPPGSVTVPHPNIEEVALSTTGEIPFYGKAIPLEAIKGGRHLIFCHSKKKCDELAAKLVALGVNAVAYYRGLDVSVIPASGDVVVVATDALMTGFTGDFDSVIDCNTCVTQTVDFSLDPTFTIETTTLPQDAVSRTQRRGRTGRGKPGIYRFVTPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPITKYIMTCMSADLEVVT

SEQ ID NO: 2 - protein, 635 aa, Hepatitis C virus
  MISC_FEATURE (1)..(635): Non-structural NS3 protein.
APITAYSQQTRGLLGCIITSLTGRDKNQVDGEVQVLSTATQSFLATCVNGVCWTVYHGAGSKTLAGPKGPITQMYTNVDQDLVGWPAPPGARSMTPCTCGSSDLYLVTRHADVVPVRRRGDSRGSLLSPRPISYLKGSSGGPLLCPSGHVVGIFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGIEPNIRTGVRTITTGGPITYSTYCKFLADGGCSGGAYDIIICDECHSTDSTTILGIGTVLDQAETAGARLVVLATATPPGSITVPHPNIEEVALSNTGEIPFYGKAIPIEAIKGGRHLIFCHSKKKCDELAAKLTGLGLNAVAYYRGLDVSVIPTSGDVVVVATDALMTGFTGDFDSVIDCNTCVTQTVDFSLDPTFTIETTTLPQDAVSRAQRRGRTGRGRSGIYRFVTPGERPSGMFDSSVLCECYDAGCAWYELTPAETSVRLRAYLNTPGLPVCQDHLEFWESVFTGLTHIDAHFLSQTKQAGDNLPYLVAYQATVCARAQAPPPSWDQMWKCLIRLKPTLHGPTPLLYRLGAVQNEVTLTHPITKYIMACMSADLEVVTSTWV

SEQ ID NO: 3 - protein, 528 aa, Artificial Sequence
  Synthetic Construct
GREILLGPADGMASKGWRLLAPITAYAQQTRGLLGCIITSLTGRDKNQVEGEVQIVPTAAQTFLATCINGVCWTVYHGAGTRTIASPKGPVIQMYSNVDKDLVGWPAPQGSRSLAPCTCGSSDLYLVTKHADVIPVRRRGDSRGSLLSPRPISYLKGSSGGPLLCPVGHAVGIFRAAVCTRGVAKAADFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCAGGAYDIIICDECHSTDATSILGIGTVLDQGETAGAKLVVFATATPPGSVTVPHPNIEEVALSTTGEIPFYGKAIPLEVIKGGRHLIFCHSKRKCDELATKLVAMGINAVAYYRGLDVSVIPTSGDVVVVATDALMTGYTGDFDSVIDCNTCVTQTVDFSLDPTFTIETTTLPQDAVSRTQRRGRTGRGKPGIYRFVAPGERPSGMFDSSVLCECYDAGCAWYELTPAE

SEQ ID NO: 4 - protein, 10 aa, Hepatitis C virus
  MISC_FEATURE (1)..(10): Epitope 1038-1047 corresponding to the NS3 viral protein
GLLGCIITSL

SEQ ID NO: 5 - protein, 9 aa, Hepatitis C virus
  MISC_FEATURE (1)..(9): Epitope 1073-1081 corresponding to the NS3 viral protein
CVNGVCWTV

SEQ ID NO: 6 - protein, 10 aa, Hepatitis C virus
  MISC_FEATURE (1)..(10): Epitope 1406-1415 corresponding to the NS3 viral protein
KLVALGINAV

SEQ ID NO: 7 - protein, 9 aa, Hepatitis C virus
  MISC_FEATURE (1)..(9): Restriction epitope h-2 Db 1629-1637
GAVQNEVTL

(19) United States
(12) Patent Application Publication
(10) Pub. No.: US 2014/0056943 A1
(43) Pub. Date: Feb. 27, 2014

(54) IMMUNO-STIMULANT COMBINATION FOR PROPHYLAXIS AND TREATMENT OF HEPATITIS C

(71) Applicant: PROYECTO DE BIOMEDICINA CIMA, S.L., Cizur Mayor (Navarra) (ES)

(72) Inventors: Aintzane ZABALETA AZPIROZ, Pamplona (Navarra) (ES); Francisco BORRAS CUESTA, Pamplona (Navarra) (ES); Jesus PRIETO VALTUENA, Pamplona (Navarra) (ES); Pablo SAROBE UGARRIZA, Navarra (ES); Juan Jose LASARTE SAGASTIBELZA, Navarra (ES)

(73) Assignee: PROYECTO DE BIOMEDICINA CIMA, S.L., Cizur Mayor (Navarra) (ES)

(21) Appl. No.: 14/039,269

(22) Filed: Sep. 27, 2013

Related U.S. Application Data
(62) Division of application No. 12/083,217, filed on Jul. 14, 2008, now abandoned.

(30) Foreign Application Priority Data
Oct. 7, 2005 (ES) P200502446
Jun. 9, 2006 (ES) P200601563

(51) Int. Cl. A61K 39/29

(52) U.S. Cl. CPC A61K 39/29
USPC 424/228.1

(57) ABSTRACT

The present invention relates to an immuno-stimulant combination for prophylaxis and treatment of hepatitis C, characterised in that it comprises: a TLR3 agonist, a CD40 agonist and the NS3 protein of the hepatitis C virus. Moreover, the invention relates to the pharmaceutical compositions comprising said immuno-stimulant combination, to the use thereof, and to a kit composed of said pharmaceutical compositions. Finally, the present invention relates to a method for producing an immune response to the hepatitis C virus and to a vaccine against said virus.

### TECHNICAL FIELD OF THE INVENTION

The present invention relates to an immuno-stimulant combination for prophylaxis and treatment of hepatitis C, which incorporates the NS3 protein of HCV, together with adjuvants selected for their capacity to induce specific potent and lasting CD8+ and CD4+ responses against the HCV virus.

### STATE OF THE ART

With an estimated world prevalence of over 170 million people infected, infection by the hepatitis C virus (HCV) today implies a heavy burden for public health. And this is a prevalence that will presumably remain invariable in the coming years.

Infection by HCV is characterised by a high tendency towards chronicity. HCV persists in 70% of infected individuals, 20% of whom develop cirrhosis and 2.5% evolve to producing cancer of the liver.

The current reference therapeutic tool is therapeutic protocols based on the use of interferon. Nevertheless, these antiviral therapies are economically costly, relatively toxic and only effective in 50-60% of patients treated. It is therefore necessary and desirable to develop new therapeutic strategies that are more effective and better tolerated by patients.

An updated review of HCV can be found in Nature (“Insights: Hepatitis C”. Nature 2005, Supplements; Vol. 436, Nr. 7053, pp 929-978).

Although, regrettably, we do not yet have an effective vaccine against hepatitis C virus, there is experimental data and evidence that leads one to think that an effective vaccine is possible. Although antiviral antibodies are synthesised in response to the infection, the chronic state is characterised by the absence of cellular immune responses on the part of cytotoxic T-cells (CD8+) and helper T-cells (CD4+). So, it is postulated that the HCV has developed strategies permitting it to specifically evade the antiviral immune responses, where the power and quality of the cytotoxic T and helper T responses determine whether the patients will recover (either spontaneously or in response to a treatment) or whether they will develop a chronic infection.

The main objective of any vaccine is to stimulate the antigen specific acquired immunity, the mediators of which are the B and T-Lymphocytes. In this context, the antigen presenting cells (APCs) play an important role in the initiation of the specific immune responses and in particular in the activation of T-Lymphocytes. APCs, mainly dendritic cells, capture antigens at the peripheral organs and, after receiving an activation stimulus, they migrate to the lymphatic organs. There, the dendritic cells do present at their surface, joined to actual molecules of the major histocompatibility complex MHC, the peptide products derived from the degradation of the antigens (epitopes), and they simultaneously produce chymokines and cytokines in order to attract and activate T-cells. The activation process of dendritic cells, also known as maturation, is characterised by a high expression of MHC molecules (signal 1), co-stimulator molecules (signal 2) and polariser cytokines such as interleukin-12 (IL-12) (signal 3). The maturation is induced by factors such as pathogen components or molecules of the host that are frequent in inflammation or cell damage processes. These factors act on the dendritic cells via specific receptors for products derived from microorganisms, such as TLR type receptors (Toll-like receptors), receptors for cytokines (TNF-α, IL-1, IFN-α) or receptors for ligands on the cell surfaces (e.g., CD40).

Stimulation and activation of the different populations of T-cells by the APCs is restricted by the type of MHC molecules on the one hand, and, on the other, by the characteristics of the epitopes which form complexes with those MHC molecules. So, for example, certain fragments has been identified of viral proteins which specifically induce the activation of cytotoxic CD8+ T-Lymphocytes (CTL), known as lymphocyte epitopes or CD8+ T-cells or CD8+ epitopes; or epitopes which specifically induce the activation of CD4+ helper T-Lymphocytes (HTL), CD4+ epitopes. The database “HCV Immunology Database” (http://hcv.lanl.gov/content/immuno/immuno-main.html) compiles the epitopes for T-Lymphocytes, both of CD8+ CTL and of CD4+ HTL, identified on the basis of viral proteins of different strains and isolates of the hepatitis C virus.

The development of immunisation protocols based on the use of epitopes in the form of peptides thus requires the previous selection of those peptides that are suitable for each individual, depending on the MHC molecules they present. This implies that, depending on the MHC of each individual, a particular combination of peptides would have to be chosen which would be able to behave as epitopes in that context. The use of large antigens permits this problem to be overcome, since they are normally polyepitopic and within their sequence they present various epitopes, both for CD8+ CTL and for CD4+ HTL, which can be presented by MHC molecules of different individuals. In this way, a single antigen can be used as a vaccine in individuals with different MHC.

Within the different proteins of HCV, core and NS3 present great immunogenicity and in those individuals which get over the infection, potent CD8+ CTL and CD4+ HTL responses are detected against them. Nevertheless, there exist data which show that core can also have deleterious effects for the cells of the immune system, when it is in contact with them, which makes it inadvisable as an antigen in vaccination strategies. On the other hand, NS3 is a protein that has scarcely demonstrated this type of effect and could be a good candidate as an antigen for induction of CD8+ CTL and CD4+ HTL responses.

The CD4+ HTL play a role in acquired immunity, among other mechanisms by means of APC activation, CTL activation and memory induction. In particular, it has been described that the CD4+ cells specific for HCV are necessary for maintenance of antiviral CTL (Grakoui A. et al., “HCV persistence and immune evasion in the absence of memory T-cell help”; Science, 2003; 302: 659-662). Therefore, an effective vaccine against the hepatitis C virus has to provide the maximum power in the induction of not just CD8+ CTL responses but also of CD4+HTL responses. Such a vaccine will therefore require a selection of specific antigens that will provide those responses.

Nevertheless, it does not seem that a combination of antigens can, on its own, be capable of providing an effective vaccine against HCV. Given that the maturation of dendritic cells is a requirement for the effective initiation and activation of T-Lymphocytes, such a vaccine could benefit from the inclusion into the immuno-stimulant combination of some adjuvants, which would stimulate the maturation of the dendritic cells. As adjuvants, use could be made of ligands of TLR receptors, of cytokine receptors or of receptors for intercellular ligands already cited, or better yet a synergic combination of those adjuvants.

So, for example, US2004/0141950 describes immuno-stimulant combinations which include an antagonist of TLRs and an antagonist of molecules of the superfamilies of the tumour necrosis factor (TNF) or of its receptors (TNFR), which can also include an antigen. Among the numerous possible combinations it presents the combination of a ligand of CD40 (an anti-CD40 antibody) and of poly(I:C), a synthetic ligand of TLR3, a combination for which a synergic effect is demonstrated in the expansion of CD8+ T-Lymphocytes. Likewise, Ahonen et al. (J. Exp. Med. 2004; 199: 775-784) present data on the synergic capacity of TLR/CD40 agonists for inducing the expansion and differentiation of antigen specific CD8+ CTL in a manner that is independent of CD4+ T-Lymphocytes. Although these works describe the capacity of the TLR/CD40 for activating CD8+ T-Lymphocytes of antigen specific memory, said works do not permit it to be established whether the combination of TLR/CD40 agonists can also boost the CD4+ HTL responses.

In the case of infection by HCV, clear differences have been found in the CD4+ HTL responses when infected patients are compared to patients who have been able to eliminate the infection. Nevertheless, although with lesser intensity than in cured patients, CD8+ CTL responses are still detectable in infected patients. Therefore, although the CTL behave as an important effector population in clearing up HCV infection, the CD4+ cells also play an important role in controlling the disease. Moreover, it has been described that the induction of CD4+ T-Lymphocytes is important for maintenance of the antiviral CTL responses (Grakoui A. et al., “HCV persistence and immune evasion in the absence of memory T-cell help”; Science, 2003; 302: 659-662). These data suggest that for the vaccination and therapy of viral diseases due to HCV, the induction of potent and lasting antiviral responses, both CD8+ and CD4+, are important.

It is therefore the object of the present invention to select immuno-stimulant combinations of antigens and adjuvants suitable for the prophylaxis and treatment of hepatitis C, which will provide a stimulation of both CD8+ and CD4+ responses that are more potent, complete and lasting.

### DETAILED DESCRIPTION OF THE INVENTION

A first object of the invention relates to an immuno-stimulant combination for prophylaxis and treatment of hepatitis C, hereinafter referred as the inventive immuno-stimulant combination, which comprises a TLR3 agonist, a CD40 agonist or a sequence of DNA that codes it, and a polypeptide which comprises the NS3 protein of the hepatitis C virus, or a fragment of said NS3 protein with capacity for inducing CD8+ and CD4+ responses.

A “TLR3 agonist” refers to a ligand which can be combined or joined to the TLR3 receptors (“toll like receptor 3”) and produce a cellular response. TLR3 is a receptor for double stranded RNA which transmits signals that activate NF-κB and the production interferons (IFN) of type I (IFN-α and IFN-β) and which stimulate the maturation of the dendritic cells. Mice lacking TLR3 expression showed a reduction in their responses to poly(I:C)—a TLR3 ligand similar to double stranded RNA generated during the replication of virus of the HCV type—, along with resistance to the lethal effect of poly(I:C) when sensitised with D-galactosamine and a reduction in the production of inflammatory cytokines (Alexopoulou et al. Nature, 2001, Vol. 413, pp. 732-738). In a particular embodiment of the invention, said ligand of TLR3 can be a viral double stranded RNA or a double chain of polyinosinic-polycytidylic acid, poly(I:C).

A “CD40 agonist” refers to a ligand, which can be combined or joined to the CD40 receptors likewise inducing a cellular response. CD40 is a molecule expressed in the membrane of different cell types, such as B-Lymphocytes or antigen presenting cells (macrophages, dendritic cells, etc.). The natural ligand of CD40 (CD40L or CD154) is mainly expressed in T-Lymphocytes which have been activated following recognition of the antigen. The interaction of CD40L with CD40 present in the antigen presenter cell induces the maturation of the latter. This phenomenon, in a way similar to the stimuli coming from pathogens, causes the antigen-presenting cell to have a greater capacity for inducing immunitary responses. So, the CD40 agonist of the inventive immuno-stimulant composition refers on the one hand to the CD40L ligand or to a fragment of that CD40L which conserves the capacity for joining to CD40 and inducing a cellular or immune response. In a particular embodiment, the ligand can be a specific antibody to CD40 (anti-CD40) or a fragment thereof which conserves the capacity for joining to CD40. Moreover, the CD40 ligand or its fragment can be present in the immuno-stimulant combination either in the form of protein or also as a recombinant nucleic acid (DNA) which codes that ligand, for example in a viral vector for transference or gene therapy.

An “antigen” refers to any substance which is capable of inducing an immune response, both humoral and cellular, in the organism of an individual (man or an animal), or which can induce a cellular immune response (expansion, activation and/or maturation of immune cells, production of cytokines, or antibodies) when it comes into contact with immunitary cells. In particular, an antigen can be a viral protein, a peptide or a fragment of said viral protein, a recombinant protein of such viral proteins or even a synthetic peptide capable of inducing the signalled responses.

A “CD8+ inducer epitope” refers to a fragment or partial polypeptide chain of an antigen that is capable of specifically inducing the activation of CD8+ cytotoxic T-Lymphocytes (CTL). A “CD4+ inducer epitope” refers to a fragment of partial polypeptide chain of an antigen that is capable of specifically inducing the activation of CD4+ helper T-Lymphocytes (HTL).

“NS3 protein” refers to the non-structural protein NS3 of the hepatitis C virus, a protein of 67 kDa which includes 2 domains, a serin-proteinase covering 189 amino acids of the N-terminal end and a domain with helicase-nucleoside triphosphatase activity covering 442 amino acids of the C-terminal end. The sequence of the NS3 protein included in the polypeptide of the inventive immuno-stimulant combination can correspond to any strain or isolate of the hepatitis C virus, in particular any strain or isolate of the human hepatitis C virus. In a particular embodiment, the polypeptide, which comprises the NS3 protein, has been obtained by recombinant technology. In a specific non-limiting embodiment of the invention, a recombinant NS3 protein is used with a sequence SEQ ID. NO: 1 (corresponding to Genebank Accession numbers DQ068198.1 and AAY84763.1, VRL 28-NOV-2005). We have also used another recombinant protein sequence SEQ ID. NO: 2 (corresponding to Genebank Accession number D90208).

In another alternative embodiment of the invention, it is possible to also use a polypeptide, which comprises a fragment of the protein NS3, in such a way that said fragment is capable of inducing CD4+ and CD8+ responses. Therefore, said fragment will have to include at least one CD8+ inducer epitope and one CD4+ inducer epitope.

In a specific embodiment, the inventive immuno-stimulant combination comprises poly(I:C), an anti-CD40 antibody, and a polypeptide containing the NS3 protein.

In a preferred embodiment of the invention, the immuno-stimulant combination possesses all the components forming part of the same pharmaceutical composition, where each one of the components is present in pharmaceutically acceptable quantities. Furthermore, the invention also refers to said pharmaceutical composition.

In another specific embodiment of the present invention, the components of the immuno-stimulant combination are to be found forming part of at least two pharmaceutical compositions. Likewise, the invention refers to the use of said immuno-stimulant combination characterised in that said pharmaceutical compositions are administered simultaneously. In another embodiment of the invention, the use of said immuno-stimulant combination is characterised in that said pharmaceutical compositions are administered at different moments, via the same administration route or via different routes. So, one specific embodiment of the invention refers to a kit for the administration of the immuno-stimulant combination described above, characterised in that it comprises at least two different pharmaceutical compositions.

In another aspect, the invention refers to a method for producing an immune response to the hepatitis C virus characterised in that it consists of administering a stimulating combination defined above, in an effective quantity for inducing an immune response. In a preferred embodiment, the method of the invention consists of a prophylactic treatment. In a more preferred embodiment, the method of the invention consists of a therapeutic treatment.

Finally, the invention also refers to a vaccine against hepatitis C virus, characterised in that it comprises an immuno-stimulant combination defined above and forming the object of this invention.

### BRIEF DESCRIPTION OF THE FIGURES

FIG. 1. Immunisation with anti-CD40 and poly(I:C) together with the NS3 protein induces multi-epitopic CD4+ and CD8+ T responses. HHD mice (two per group) were injected with 50 g of anti-CD40 (i.p.). Four hours later, they were injected with 50 g of poly(I:C) (i.v.) and 500 g of recombinant NS3 protein (i.p.) (SEQ. ID. NO: 1). Six days later, the animals were killed and the splenocytes were extracted for their in vitro stimulation with different antigens and the analysis of the induced immunitary response. (A) The cells were stimulated for five days with the epitopes CD8+ 1073, 1406 or 1038 (10 μM) in the presence of IL-2. Afterwards, for each group of splenocytes, the Iythic response was measured to target cells that were loaded (peptide; black bars) or not (control; white bars) with the corresponding peptide. The results obtained were shown with an effector:target ratio of 100:1. B) In the same way, the splenocytes were cultured with different concentrations (0.1-10 μM) of the peptides 1073 (black circles), 1406 (white triangles) or 1038 (black triangles), and in the culture supernatants obtained after 48 h of stimulation the IFN-γ content was measured by means of ELISA. (C) The splenocytes were also stimulated for 48 h with 5 or 1 μg/ml of the NS3 protein used in the immunisation (SEQ. ID. NO: 1), with 1 μg/ml of the NS3 protein produced in bacteria (SEQ. ID. NO: 3), or with culture medium (control) in order to measure the CD4+ response. Following this period of time the supernatants were collected and the amount of IFN-γ produced was measured by means of ELISA.

FIG. 2. Measurement of the quantity of NS3 protein necessary for inducing CD4+ and CD8+ T responses in immunisation with poly(I:C) and anti-CD40. HHD mice (two per group) were immunised with NS3 protein (SEQ. ID. NO: 1) (500, 250, 125 or 25 μg/mouse) together with poly(I:C) and anti-CD40, following the protocol described in FIG. 1. Also included was a control group immunised in the same way, which used as antigens 5 μg of NS3 (SEQ. ID. NO: 1) and 50 μg of the peptides 1073 and 1038, along with poly(I:C) and anti-CD40. Six days later the animals were killed and the splenocytes were extracted and stimulated with different antigens (A). In order to measure the induced Iythic response the cells were stimulated for five days with the epitope CD8+ 1073 (10 μM) and IL-2. Afterwards, that response was measured by confronting different quantities of effector cells against a fixed number of target cells loaded with the peptides. Moreover, the CD8+ response that had been induced was also analysed by means of the production of IFN-γ. To do this, the cells were stimulated with different concentrations of peptides 1073 (B) and 1038 (C). The cells were also stimulated with the NS3 protein (SEQ. ID. NO: 1) (D), in order to quantify the CD4+ response. After 48 h, the amount of IFN-γ present in the supernatants was measured.

FIG. 3. Immunisation with poly(I:C) and anti-CD40 together with the NS3 protein induces CD4+ and CD8+ responses in other strains of mice with different MHC. C57BL6 mice (which have MHC molecules of the type H-2b) (two per group) received one (white squares) or two (black squares) immunisations with 100 μg of NS3 (SEQ. ID. NO: 1) together with poly(I:C) and anti-CD40 following the protocol indicated in FIG. 1. Six days later, the animals were killed and the splenocytes were cultured with different antigens in order to measure the induced CD8+ and CD4+ responses. The restriction epitope H-2 Db 1629-1637 (GAVQNEVTL) (SEQ. ID. NO: 7) was used for stimulating the splenocytes and measuring CD8+ responses (A). The NS3 protein (SEQ. ID. NO: 1) (B) was used as stimulus for determining the CD4+ response. After two days of culture, the supernatants were collected and the amount of IFN-γ produced was measured.

FIG. 4. Immunisation with NS3 protein together with poly(I:C) and anti-CD40 induces CD8+ responses capable of recognising cells that express proteins of the HCV. (A) HHD mice (two per group) were injected with 100 μg of NS3 protein (SEQ. ID. NO: 2) plus poly(I:C) and anti-CD40 as indicated in FIG. 1. Six days later, the animals were killed and their splenocytes were stimulated with T1/HCVcon cells (T1 cells transfected with a plasmid that expresses the proteins of the HCV) treated with mitomycin, in the presence of IL-2. After five days of stimulation, the capacity of the splenocytes to recognise the T1/HCVcon cells was measured in Iythic activity assays. To do this, different quantities of splenocytes were confronted with a fixed number of T1/HCVcon cells (black circles) or T1 control cells without being transfected (white circles).

FIG. 5. Immunisation with poly(I:C) and anti-CD40 together with NS3 protein induces lasting T CD4+ and CD8+ responses. HHD mice (two per group) were injected with 100 μg of NS3 protein (SEQ. ID. NO: 2) plus poly(I:C) and anti-CD40 as indicated in FIG. 1. Two weeks later, the animals received a second immunisation under the same conditions. Sixty days after the second immunisation the animals were killed and their splenocytes were extracted for studying the lasting CD8+ and CD4+ T response. (A) The splenocytes were stimulated with the epitope CD8+ 1073 (10 μM) or in the absence of antigen, and 48 hours later the culture supernatants were collected for measuring the amount of IFN-γ produced. (B) The splenocytes were cultured for 5 days with the peptide 1073 (10 μM) and IL-2 and their capacity to lyse target cells loaded with the peptide 1073 was then studied. To do this, different quantities of effector cells were confronted with a fixed number of target cells loaded with the peptide 1073 (black circles) or without loading with peptide (white circles). (C) The CD4+ response was studied by means of stimulation of the splenocytes with the NS3 protein (1 μg/ml) (SEQ. ID. NO: 2) or in the absence of antigen. After 48 hours, the supernatants were collected and the amount of IFN-γ produced was measured.

### MODE OF EMBODIMENT OF THE INVENTION

The following examples, without in any way being limiting, aim to illustrate the embodiment of the invention forming the present patent application.

### RELATIVE MATERIAL AND METHODS

### Epitopes, Antigens and Reagents

The peptides or epitopes used were synthesised manually in a multiple peptides synthesiser using Fmoc chemistry (Wellings D A. and Atherton E. Methods Enzymol 1997; 289: 44-67). The Kaiser ninhydrine test was used for monitoring each step. At the end of the synthesis they were spliced and deprotected with trifluoroacetic acid and washed with diethyl ether. The purity of the peptides was at all times higher than 90% determined by HPLC.

TABLE 1
Peptides and epitopes synthesised and used in the examples.

| Peptide or | |
|---|---|
| Epitope | Sequence |
| 1038-1047 | GLLGCIITSL; SEQ. ID. NO: 4 |
| 1073-1081 | CVNGVCWTV; SEQ. ID. NO: 5 |
| 1406-1415 | KLVALGINAV; SEQ. ID. NO: 6 |
| 1629-1637 | GAVQNEVTL; SEQ. ID. NO: 7 |

The numbering of the peptide or epitope refers to its relative HCVH position, taking as reference the complete sequence in the H strain of human hepatitis C which is usually taken as the prototype (GeneBank Accession Number M67463). So, for example, the database “HCV Immunology Database” (http://hcv.lanl.gov/content/immuno/immuno-main.html) compiles the epitopes for T-Lymphocytes, both of cytotoxic T-Lymphocytes and of helper T-Lymphocytes, identified in the viral proteins of different strains and isolates of the hepatitis C virus, all of them also ordered in accordance with their relative position with respect to the H strain of the virus according to the stated GeneBank reference.

As immunogen, a recombinant polypeptide of 655 amino acids has been used which contains the complete sequence of the NS3 protein (SEQ. ID. NO: 1; Genebank accession number AAY84763.1, VRL 28-NOV-2005; 631 amino acids). As well as the 631 amino acids of the NS3 protein, the polypeptide also includes a tail with a c-myc sequence, for detection with the monoclonal antibody anti-myc, and a tail of Histidines. The protein has been produced in Pichia pastoris. It is maintained in suspension in a solution of Tris 22.5 mM/Urea 3.76 M/NaCl 300 mM. The protein has been purified by means of Ni column chromatography.

Another recombinant polypeptide has also been used as immunogen, which contains the 635 amino acids comprising the complete sequence of the NS3 protein (SEQ. ID. NO: 2; Genebank accession number D90208). As well as the amino acids corresponding to NS3, the polyprotein also includes a tail of Histidines for its purification. The DNA sequence corresponding to NS3 was obtained by digestion with Sal I and Not I of the plasmid gWIZ, which contained the NS3, sequence (supplied by Dr. G. Inchauspe, Lyon, France). The product of the digestion was cloned between the sites BsrG I and Not I of the plasmid pET-45 (+) (Novagen, Madison Wis.). It was expressed with E. coli and purified by means of affinity chromatography in a nickel column followed by ion exchange chromatography.

Likewise, for the in vitro assays a recombinant polypeptide (Mikrogen; Catalogue number 94302) has been used as antigen, which contains the last 20 amino acids of the non-structural protein NS2 and the first 508 amino acids of the NS3 protein of HVC (SEQ. ID. NO: 3).

As TLR3 agonist, poly(I:C) has been used obtained from Amersham (Catalogue number 27-4732-01).

As CD40 agonist, anti-CD40 antibodies were used, purified starting from the hybridome FGK-45 (Rolink A. et al., Immunity 1996. 5: 319-330).

All the reagents contained <1 unit of endotoxin per mg of product, determined by means of the lysate QCL-1000 assay of the amoebocyte limulus (Bio Whittaker).

Mice

C57B1/6 mice of six to eight weeks were obtained from Harlan. HHD mice were also used, transgenic for human molecules HLA-A2.1 (Pascolo S. et al., J. Exp. Med. 1997. 185: 2043-2051). All the animals were maintained under pathogen free conditions and were treated in accordance with the rules of the institution.

Cell Lines

T2 cells were used (Salter R. et al. Immunogenetics, 1985 21: 235-246) as target cells for chromium release assays with cytotoxic T-Lymphocytes (CTL) coming from HHD mice.

T1 cells were used, transfected with a carrier plasmid of the coding region of the HCV (T1/HCVcon cells), for the recognition assays of cells which expressed the proteins of the HCV. These cells were provided by Dr. D. Moradpour (Freiburg, Germany; Volk B. et al., J Gen Virol. 2005; 86: 1737-1746). T1 cells without transfecting (ATCC, catalogue Nr. CRL-1991) were also used as control.

All the cells were grown in complete medium (RPMI 1640 10% of foetal bovine serum, 100 U/ml of penicillin, 100 μg/ml of streptomycin, 2 mM of glutamine and 50 μM of 2-mercaptoethanol). The culture of the line T1/HCVcon also contained 2 mg/ml of G418 (Gibco).

Immunisation

Groups of two mice were immunised via the i.p. route with 50 μg of anti-CD40. Four hours later, they were injected with 50 μg of poly(I:C) (i.v.) and different amounts of the antigens: NS3 protein or mixtures of NS3 with peptides (i.p.).

### Stimulation of Splenic Cells for the Production of Cytokines

Splenic cells were resuspended in complete medium and plated at 8×105 cells/well in 0.2 ml on 96-well plates with U-shaped bottom, in the absence or presence of peptides or of the recombinant NS3 protein of the HCV.

Two days afterwards, the supernatants were collected for measuring the presence of IFN-γ by means of ELISA (BD-Pharmingen), following the manufacturer's instructions.

### Measurement of the Iythic Activity of CTL

In order to measure the CTL responses, the splenocytes coming from the immunised animals were incubated with peptides (10 μM) for 2 h at 37° C., washed twice and cultured on 24-well plates with a confluence of 7.5×106 cells/well. In experiments conducted for measuring the recognition of T1/HCVcon cells, 7.5×106 splenocytes of HHD mice were cultured with 7.5×105 T1/HCVcon cells previously treated with Mitomycin C (Sigma). In all cases, two days later, 2.5 U/ml of IL-2 (Boehringer-Mannheim GmbH, Germany) was added to the wells and 5 days later the cells were recovered in order to carry out chromium release assays.

The Iythic activity was measured by incubating different quantities of effector cells for 4 h with 3000 T2 target cells previously loaded with 51Cr, with and without peptide (target). In the case of cells stimulated with T1/HCVcon, the effector cells were confronted with T1/HCVcon or T1, previously loaded with 51Cr. The culture supernatants were collected after 4 h of incubation.

The specific lysis percentage was calculated according to the formula:

(cpmexperimental−cpmspontaneous)/(cpmmaximum−cpmspontaneous)×100

where the spontaneous lysis (measured as cpmspontaneous) corresponds to target cells incubated in the absence of effector cells, and the maximum lysis (cpmmaximum) is obtained by incubating target cells with 5% Tritonx100.

### Example 1

Immunisation with Anti-CD40 and Poly(I:C) Together with the NS3 Protein Induces Multi-Epitopic CD4+ and CD8+ T Responses.

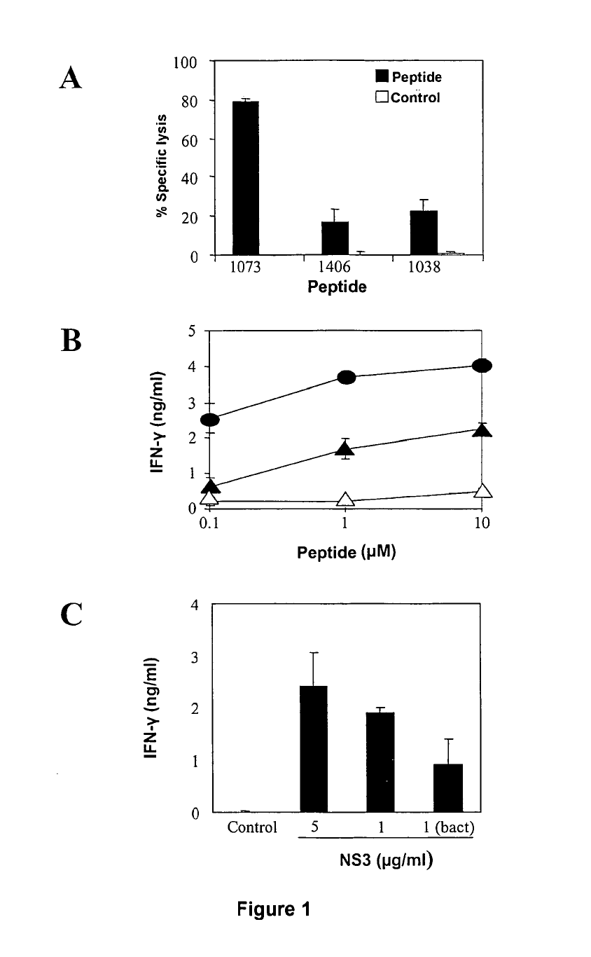

Immunisation with anti-CD40 and poly(I:C) has shown itself to be very effective for the induction of CD8+ responses by means of using as immunogens synthetic peptides which represent epitopes of CD8+ cells. Although this strategy induces potent responses, it has been demonstrated that when it is co-immunised with low quantities of NS3 protein (5 μg/mouse), which induces CD4+ response, it increases the magnitude of the CD8+ response and it also increases the high affinity CD8+ response, in other words, the one which recognises low concentrations of antigen. Moreover, immunisation with peptides is only effective in those individuals who possess HLA molecules of the same restriction as the chosen epitopes. With the aim of tackling these two points, a study was made of whether immunisation with greater quantities of recombinant NS3 protein would be capable of inducing responses, not just CD4+ but also CD8+. To do this, mice were immunised with NS3 along with poly(I:C) and anti-CD40, and the induced responses were studied. So, HHD mice (two per group) were injected i.p. with 50 μg of anti-CD40. Four hours later, they were injected with 50 μg of poly(I:C) (i.v.) and 500 μg of recombinant NS3 protein (i.p.) (SEQ. ID. NO: 1). Six days later, the animals were killed and the splenocytes were extracted. With the aim of analysing the NS3 capacity, when the adjuvant poly(I:C)+anti-CD40 is formulated to induce CD8+ and CD4+ T responses, the splenocytes were stimulated in vitro with different antigens which specifically activates these cell populations. (A) In order to analyse the CD8+ response, in a first experiment the splenocytes were stimulated for five days with the epitopes CD8+ 1073, 1406 or 1038 in the presence of IL-2. Afterwards, for each group of cells stimulated with a peptide, their capacity was measured to lyse to target cells that were loaded with the corresponding peptide (black bars) or to control target cells without peptide (white bars). FIG. 1A shows the results obtained with an effector:target ratio of 100:1. (B) The CD8+ response induced after immunisation with NS3 was also analysed by means of studying the production of IFN-γ towards the same CD8+ epitopes. To do this, the splenocytes were cultured with different quantities of 1073 (black circles), 1406 (white triangles) or 1038 (black triangles). After 48 h of culture, the supernatants were collected and the IFN-γ content was measured. (C) With the aim of analysing the induced CD4+ response, the splenocytes were stimulated with the NS3 protein used in the immunisation (SEQ. ID. NO: 1). Also, the cells were stimulated with commercial NS3 protein produced in bacteria (SEQ. ID. NO: 3). In the same way as in the previous point, the degree of activation was measured by means of the production of IFN-γ.

First of all, it was possible to check that this antigen was capable of inducing CD8+ responses, which could be detected both in chromium release assays (FIG. 1A) and by means of the induction of the production of IFN-γ (FIG. 1B). Moreover, this response was multi-epitopic, being directed towards various CD8+ epitopes, which have been characterised within the NS3 sequence (e.g.: peptides 1073, 1406 and 1038). Finally, it was also confirmed that it was capable of inducing CD4+ responses, which recognised the NS3 protein used in the immunisation and the commercial NS3 protein produced in bacteria (FIG. 1C). The response towards this latter was lower, presumably due to the fact that there existed some changes in the sequence of both proteins and that the protein expressed in bacteria was shorter, with which it could lose some epitopes recognised by the CD4+ T-Lymphocytes.

### Example 2

The Administration of 25 μg of Recombinant NS3 Together with Poly(I:C) and Anti-CD40 is Sufficient for Inducing CD4+ and CD8+ T Responses.

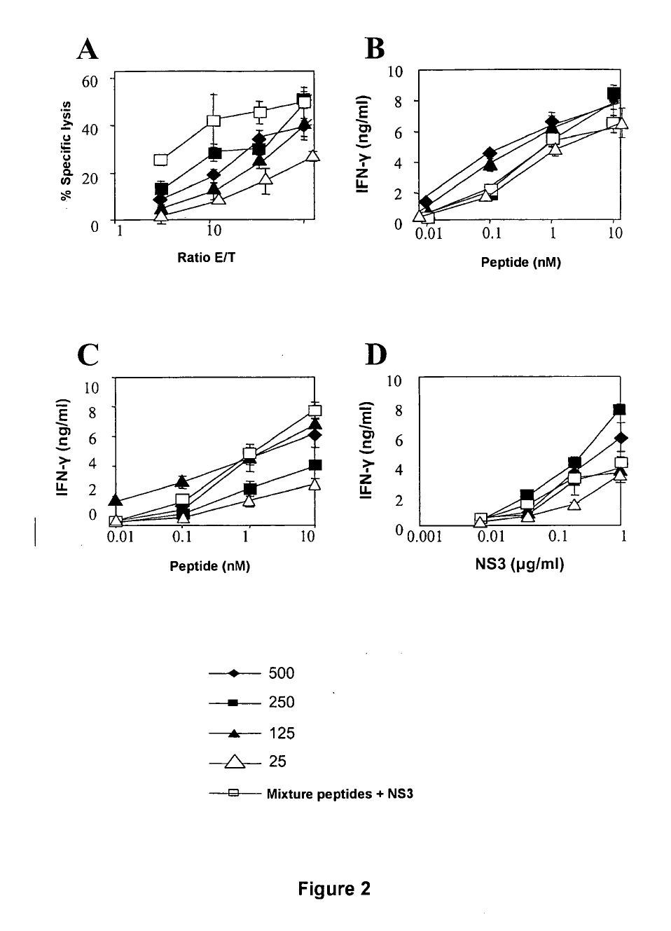

From previous experiments we knew that with 5 μg of NS3 CD4+ responses were induced but not CD8+, and we therefore wished to discover the minimum quantity of NS3 that would be sufficient for inducing CD8+ responses. To do this, HHD mice were immunised with 500, 250, 125 and 25 μg of NS3 (SEQ. ID. NO: 1). Also included as control was a group immunised with peptides corresponding to CD8+ epitopes, which would induce CD8+ responses, plus 5 μg of NS3 (SEQ. ID. NO: 1), which would induce CD4+ responses. For this, in each group of animals immunised with a dose of NS3 an analysis was conducted of the CD8+ response and the CD4+ response. The CD8+ response was analysed as the capacity to lyse to target cells loaded with the epitope CD8+ 1073 (FIG. 2A), along with the capacity to produce IFN-γ with regard to different concentrations of the epitopes CD8+ 1073 (FIG. 2B) and 1038 (FIG. 2C). The CD4+ responses were measured by means of the capacity to produce IFN-γ with regard to different concentrations of NS3 (SEQ. ID. NO: 1) (FIG. 2D). This experiment demonstrated that all the quantities of NS3 assayed were capable of inducing CD8+ responses, when the Iythic responses to the peptide 1073 were studied (FIG. 2A), the dose of 25 μg being the one that induced responses of the weakest intensity. Moreover, all the doses were capable of inducing the production of IFN-γ with regard to the epitopes 1073 (FIG. 2B) and 1038 (FIG. 2C), which indicated that the capacity to induce multi-epitopic responses was maintained even when the doses were reduced. Finally, and as was expected, all of them induced CD4+ responses. Given that, in the majority of cases, the induced response was less when 25 μg of NS3 was used, for later experiments a dose of 100 μg/mouse was chosen, starting from which dose no increase was observed in the induction of responses.

### Example 3

Immunisation with Poly(I:C) and Anti-CD40 Together with the NS3 Protein Induces CD4+ and CD8+ Responses in Other Strains of Mice with Different MHC.

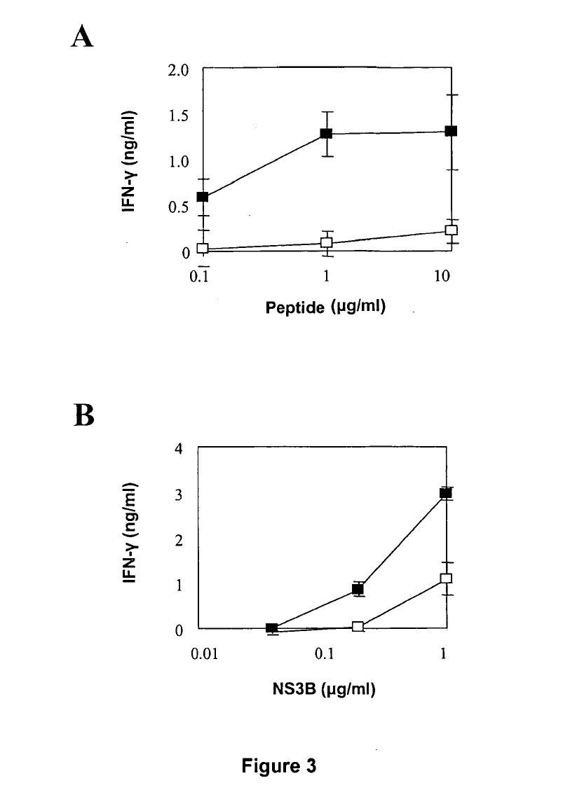

Given that in an antigen as large as the NS3 protein, it is possible to find CD4+ and CD8+ epitopes, which can be presented by different molecules of MHC, the capacity of this immunisation protocol for inducing CD4+ and CD8+ responses in another strain of mouse with different MHC molecules was studied. To do this, C57/Bl6 mice, which have H-2b restriction MHC molecules, were immunised with 100 μg of NS3 (SEQ. ID. NO: 1). With the aim of improving the responses, one group received a single immunisation and the other group received a second booster immunisation. First of all, the CD8+ response was measured, as the production of IFN-γ against the peptide 1629-1637 (SEQ. ID. NO: 7), which contains a CD8+ epitope presented by the MHC molecules of class I H-2 Db. As can be seen in FIG. 3A, a detectable response was induced in both groups of mice, though the levels were considerably greater in the group that had received two immunisations (black squares) than in the one that received one immunisation (white squares). The CD4+ response, measured as the production of IFN-γ against the recombinant NS3 protein (SEQ. ID. NO: 1) was also detected in the two groups (FIG. 3B), and again demonstrated that two immunisations (black squares) induced more potent responses that a single immunisation (white squares).

### Example 4

Immunisation with NS3 Protein Together with Poly(I:C) and Anti-CD40 Induces CD8+Responses Capable of Recognising Cells that Express Proteins of the HCV.

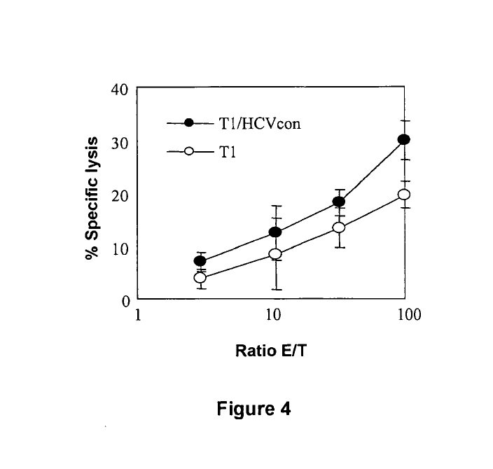

With the aim of studying whether immunisation using NS3 protein together with poly(I:C) and anti-CD40 would be capable of inducing responses that could potentially kill cells infected with HCV, an in vitro model was used of target cells transfected with a plasmid that expressed the proteins of the HCV (T1/HCVcon). These cells expressed the same peptides in their Class I MHC molecules as would be expressed by a cell infected with HCV; therefore, it could be assumed as a response against the latter any certain response against them. The NS3 protein (SEQ. ID. NO: 1) used in the experiments of FIGS. 1 to 3 corresponds to a different viral strain from the viral strain present in the T1/HCVcon cells. These two strains present some differences in the CD8+ epitopes studied so far. With the aim of optimising the recognition capacity of the CD8+ epitopes present in the T1/HCVcon cells, for this experiment an NS3 protein (SEQ. ID. NO: 2) was used as immunogen, whose sequence had a degree of homology greater than the protein present in the T1/HCVcon cells. Six days after immunisation of HHD mice with 100 μg of NS3, the splenocytes were stimulated with T1/HCVcon cells. The recognition capacity of T1/HCVcon cells was analysed in Iythic activity assays. To do this, stimulated splenocytes were confronted with T1/HCVcon cells and T1 control cells. As shown in FIG. 4, immunisation with NS3 induced responses with a greater capacity to lyse T1 cells, which expressed proteins of the HCV (black circles) than T1 control cells (white circles).

### Example 5

Immunisation with Poly(I:C) and Anti-CD40 Together with NS3 Protein Induces Lasting T CD4+ and CD8+ Responses.

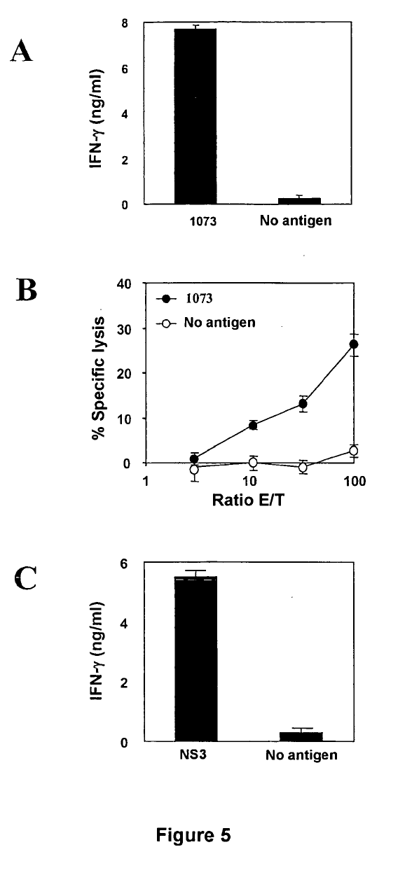

One of the main properties that a vaccination protocol has to possess is its capacity to induce lasting immunitary responses, so that the protection conferred by the immunisation can persist in the long term. In order to study whether immunisation with anti-CD40 and poly(I:C) together with the NS3 protein would be capable of inducing this kind of response, HHD mice were immunised with 100 μg of NS3 in accordance with the protocol described in example 1. With the aim of reinforcing the response, after 15 days the animals received a booster dose under the same conditions. Sixty days after the second immunisation the animals were killed and their splenocytes were stimulated with different antigens in order to analyse the CD8+ and CD4+ T responses persisting at that moment. In order to study the CD8+ T response, the cells were stimulated with the epitope 1073 and the production of IFN-γ and the Iythic activity were measured. As shown in FIG. 5A, sixty days after the second immunisation, the splenocytes of mice immunised with anti-CD40 and poly(I:C) together with the NS3 protein were capable of producing large amounts of IFN-γ when stimulated with the peptide 1073, but not in the absence of antigen. Moreover, these cells were capable of lysing target cells pulsed with the peptide 1073 (black circles) but not target cells that did not contain antigen (white circles) (FIG. 5B). Finally, the CD4+ response was also studied, using as antigen the NS3 protein used in the immunisation. FIG. 5C shows that this immunisation protocol also induces potent and lasting CD4+ responses, which specifically recognise NS3.

1-17. (canceled)

18. A method for producing an immune response to the hepatitis C virus, characterized in that it consists of administering a immuno-stimulant combination comprising:
a) poly(I:C) acting as a TLR3 agonist,
b) a CD40 agonist or a sequence of DNA that codes it, and
c) a polypeptide, which comprises the NS3 protein of the hepatitis C virus, or a fragment of said NS3 protein with capacity for inducing CD8+ and CD4+ responses,
in an effective quantity for inducing an immune response.

19. A method according to claim 18, wherein it comprises a prophylactic treatment.

20. A method according to claim 18, wherein it comprises a therapeutic treatment.
